(19) United States
(12) Patent Application Publication
(10) Pub. No.: US 2007/0159600 A1
(43) Pub. Date: Jul. 12, 2007

(54) Transcleral opthalmic illumination method and system

(75) Inventors: Tamir Gil, Kibbutz Givat Haim Meuchad (IL); Oded Wigderson, Haifa (IL); Amit Sasson, Herzelia (IL); Zvi Nizani, Nofit (IL)

Correspondence Address: BROWDY AND NEIMARK, P.L.L.C.;624 NINTH STREET, NW, SUITE 300, WASHINGTON, DC, 20001-5303, US

(73) Assignee: MEDIBELL MEDICAL VISION TECHNOLOGIES, LTD., HAIFA (IL)

(21) Appl. No.: 10/552,269

(22) PCT Filed: Apr. 8, 2004
(86) PCT No.: PCT/US04/10617

§ 371 Date: Sep. 22, 2006

Related U.S. Application Data
(60) Provisional application No. 60/460,821, filed on Apr. 8, 2003.
(60) Provisional application No. 60/515,421, filed on Oct. 30, 2003.

(51) Int. Cl. A61B 3/10

(52) U.S. Cl. 351/221

(57) ABSTRACT

A method and apparatus for illuminating the interior of the eye through the sclera without any contact to the eye. The apparatus contains a lamp element and optics that focus the light on the eye sclera. One or more fiber optics bundles may be used to convey the light from the light source close to the illuminated eye, ending with condensing optical elements. Alternatively, light could be conveyed by sharing the optics of an imaging system. It is useful for observing or imaging the interior of the eye, the retina, or the choroid. The observation or the imaging of the interior of the eye, the retina, or the choroid by applying the disclosed illumination method can be done in conjunction with any system that includes optics for that purpose, e.g., fundus cameras and ophthalmoscopes, without using those systems' illumination elements.

### BACKGROUND

The present application claims priority from U.S. Provisional Application Ser. No. 60/460,821, filed Apr. 8, 2003, and U.S. Provisional Application Ser. No. 60/515,421, filed Oct. 30, 2003. The disclosures of both these applications are incorporated by reference herein in their entirety.

### FIELD

This invention relates to ophthalmoscopes, fundus cameras, slit lamps and operation microscopes, i.e., instruments for viewing and imaging the interior of the eye. More particularly, the invention provides an illumination method serving to provide adequate illumination for diagnostic and documentation purposes of these systems, while making their operation possible without pupil dilation, while enlarging their observable field to the whole fundus, and by-passing illumination difficulties due to opacities and scattering of the anterior chamber of the eye. The observable field is the area of the fundus beyond which the observation system is unable to reach.

### PRIOR ART

Currently, most known fundus-viewing and imaging systems illuminate the interior of the eye through the pupil of the eye by a light source that is located in the region of the camera and is directed into the posterior segment of the eye. These systems suffer from reflections of the illuminating light off the cornea, crystalline lens, and its interface with the vitreous cavity. They need typically more than half of the pupil area for illumination, and when attempting to view the interior of the eye at locations more peripheral than the macula, the effective pupil size that is available becomes smaller and light does not go through. As a result, standard fundus viewing and imaging systems depend strongly on-clear ocular media and on wide pupil dilation. They are limited to a maximum of 60° field of view (FOV) and cannot observe the periphery much beyond the posterior pole. They are thus of limited use for patients with nondilating pupils, such as those with chronic glaucoma, uveitis, and diabetes mellitus, and for patients with opaque media, cataract, and pseudophakic lens.

The problems evolved in illuminating the interior of the eye through the pupil can be avoided when the interior of the eye is illuminated through the sclera (transcleral illumination), as first proposed by Pomerantzeff in U.S. Pat. No. 3,954,329. This method supports wide angle fundus imaging without demanding pupil dilation and by-passing illumination difficulties that may rise due to obstruction and scattering from opacities in the anterior eye chamber. In addition it enlarges the observable field to the whole fundus. Recently, a system (Panoret-1000™ of Medibell Medical Vision Technologies, Ltd.) that is based on U.S. Pat. No. 5,966,196 (Svetliza, et al.) and U.S. Pat. No. 6,309,070 (Svetliza, et al.) has applied transcleral illumination according to U.S. Pat. No. 3,954,329. The advantages and applicability of transcleral illumination as realized with Panoret-1000™ have recently been discussed by Shields et al. (Rev. Ophth. 10, 2003, Arch. Ophth 121, 2003). However, this system, as well as improvements that were suggested in U.S. Pat. No. 4,061,423 (Pomerantzeff), U.S. Pat. No. 4,200,362 (Pomerantzeff), and U.S. Pat. No. 6,309,070 (Svetliza, et al.), has suffered from relying on optical elements that needed to touch the sclera of the eye. Moreover, all the aforementioned systems were designed to work in conjunction with cameras that operated in contact with the eye cornea. Thus they were limited in their applicability in the general practice of ophthalmology and they were not suitable for work in conjunction with standard cameras and optics.

Touching the eye sclera requires an operator hand and extra attention, or, alternatively sophisticated mechanics. It requires local anesthetics, disinfection of the touching elements, and often the use of a speculum that helps to reveal the sclera.

According to one embodiment of the present invention, a method is provided for illuminating the interior of an eye through the sclera of the eye, comprising focusing a light beam on the sclera by focusing optics while maintaining the focusing optics out of contact with the sclera.

According to another embodiment of the present invention, a system is provided for ophthalmic illumination of the interior of the eye of a patient through the sclera of the eye without touching the eye comprising a light source, optics that focus the light from the light source to a light spot on the sclera without touching the sclera, and opto-mechanical means for directing the focused beam to a desired position on the eye sclera.

### SUMMARY

Accordingly, this invention provides a system for transcleral illumination of the eye interior, without touching the eye. Such a system eliminates the chance of damaging the eye or causing discomfort to the patient as has been heretofore. Moreover, it does not induce extra eye movements or dependence on the operator's hand stability that in contact systems give rise to a lower acquisition success rate, i.e., this invention increases the efficiency of systems that would apply transcleral illumination.

### BRIEF DESCRIPTION OF THE DRAWINGS

For a better understanding of the invention with regard to the embodiments thereof, reference is made to the accompanying drawings, in which like numerals designate corresponding elements or sections throughout, and in which:

FIG. 1 shows an example of illumination pattern on the patient eye upon transcleral illumination.

FIG. 2 is an exemplary embodiment of the illumination system of the present invention.

FIG. 3 illustrates an exemplary method of controlling the shape of the light spot on the eye sclera in the exemplary embodiment of FIG. 2.

FIG. 4 is a block diagram of the computerized controls of the illumination system of the exemplary embodiment of the present invention.

FIG. 5 is one example of how the present invention can be realized in conjunction with a standard commercial fundus camera.

FIG. 6 is another exemplary embodiment of the illumination system of the present invention.

FIGS. 7(a) and 7(b) are retinal images acquired with the system shown in FIG. 5 applying transcleral illumination.

FIGS. 8(a) and 8(b) show another example of the present invention in which the transcleral illumination spots are brought to the right position on the sclera by letting the patient position the eye.

FIG. 9 is another example of how the present invention can be realized.

FIG. 10 is another example of how the present invention can be realized in conjunction with a standard commercial fundus camera.

FIG. 11 is another example of how the present invention can be realized in conjunction with imaging optics.

FIG. 12 illustrates one embodiment of the illumination optics that serves for focusing a light spot on the eye sclera in the systems of FIGS. 10 and 11.

FIG. 13 illustrates light blocking elements that prevent light that is scattered from the sclera from reaching the observation and imaging optics according to one embodiment of the present invention.

FIG. 14 is an image of the retina acquired with the system shown in FIG. 11 while applying transcleral illumination.

FIG. 15 shows optics to illuminate the eye sclera both on the nasal and the temporal side simultaneously.

FIG. 16 is another example of how the present invention can be realized.

FIG. 17 is another example of how the present invention can be realized.

### DETAILED DESCRIPTION

The present invention overcomes disadvantages associated with the need to touch the sclera of the eye upon application of transcleral illumination for ophthalmic examination of the retina, and provides a method and apparatus that enables the application of transcleral illumination with any optics used for imaging the interior of the eye, the retina, and the choroid. As a result of this invention, transcleral illumination with its aforementioned advantages will be available for use in conjunction with existing fundus examination and imaging systems as well as with particularly designed new optics with superior fields of view and fields of observation, which operates with a non-dilated pupil. Superimposing several images from those acquired by these systems at different angles will provide a fully documented view of the entire fundus, which is currently obtained by using contact (to the cornea) cameras that are cumbersome in use and uncomfortable for the patient.

Transcleral illumination is preferably directed through a narrow region of the sclera that lies external to the pars plana and transmits light in the visible range better than other locations on the sclera. For this reason as well as because of the natural small opening gap of the eyelids and the need to prevent light from reaching the eye cornea and being reflected further into the imaging optics, it is preferred to concentrate the illuminating spot to be only a few millimeters in size and direct it to the pars plana. The present invention provides efficient means to direct it to the optimal location with the required power that is higher than the power required for the standard transpupillary illumination because of the optical properties of the sclera, which transmits less than 50% of the visible light that is shined on it.

The physical structure of the sclera is very diffusive and gives rise to relatively even spreading of the light that passes through it. This yields relatively high uniformity in the illumination of the retina. Hence, transcleral illumination supports examination of the ocular fundus by direct observation and by electronic and photographic means.

In accordance with an exemplary embodiment of the present invention there is provided a method and apparatus for non-contact transcleral illumination.

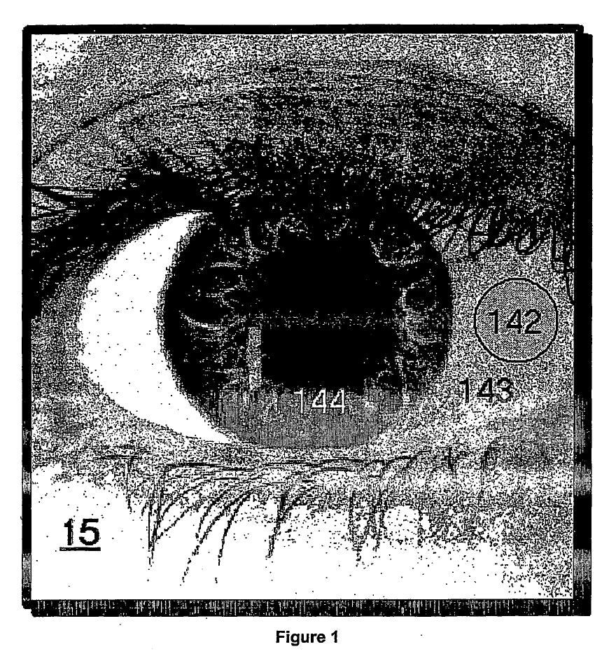

The applicability of the present inventions relies very much on two principal capabilities—one, focusing the light emitted from a light source into a small spot without losing energy, and, two, bringing the light spot efficiently to the right position on the sclera, above the pars plana. These two capabilities influence each other because the efficiency of focusing the light depends on the size of the focusing element, and the size of the focusing element influences the ability of moving it around without colliding with elements belonging to the imaging system, e.g., the fundus camera. FIG. 1 illustrates where the light spot 142 should be focused on the patient's sclera 143, on the surface of the eye 15 at about 3 millimeters from the limbus, which lies approximately above the perimeter of iris 144.

Five exemplary concepts and systems for efficiently achieving the goals of focusing the light into small spots and bringing the spots to the right location on the patient's eye, taking into account the need to allow efficient alignment and focusing of the imaging system that is applied in conjunction with the transcleral illumination, are presented in the five examples below.

The first example takes the approach of coupling between eye position and the light focusing element, i.e., fixing the head of the patient, directing its look to fix the position of the eye, and then placing the light spot at the appropriate location on the eye surface.

The second example takes the approach of letting the patient bring the eye to a designated location, directing the illumination light spots in a way that whenever the eye is in place then the light spots fall on the appropriate position on the eye sclera.

The other examples take the approach of coupling between the light focusing element and the optical imaging system, devising them in a way that the imaging system and the focused light spot will be properly positioned simultaneously.

The first example has the advantage of optimal placement of the light spot along with giving the imaging system full freedom of observing the eye from all directions. However, this positioning adds an extra step to the acquisition process in comparison to standard fundus photography, and it is very sensitive to the patient's head and eye movements during the examination process.

The second example has the advantage that the patient brings the eye herself or himself to the right position, reducing operator activities thus shortening photography time and making the system more efficient. This approach is however more sensitive to eyelids and face structure and a single device bears the risk of not fitting the entire population.

The other examples have the advantage that the operator concentrates in aligning only one system, the imaging system, while the illumination spot moves with it to its appropriate position. In these examples the position of the light spot relative the optical center of the imaging system is designed to fit an eye of average dimensions. As a result, deviations among different people may give rise to non-ideal positioning of the light spot.

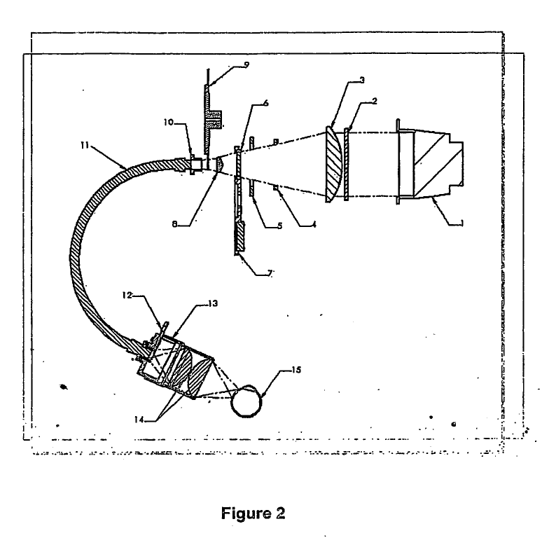

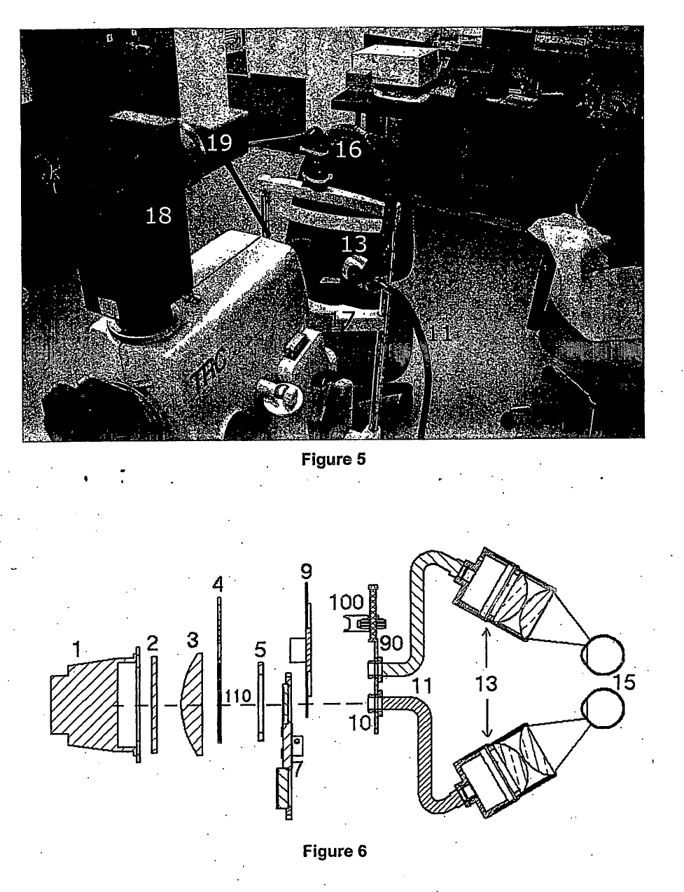

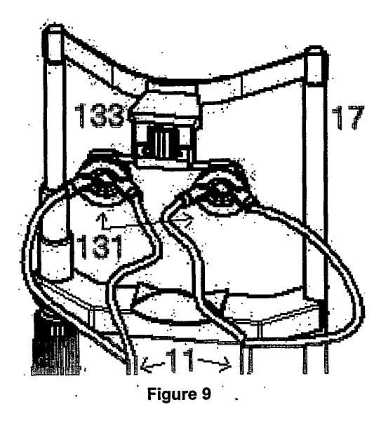

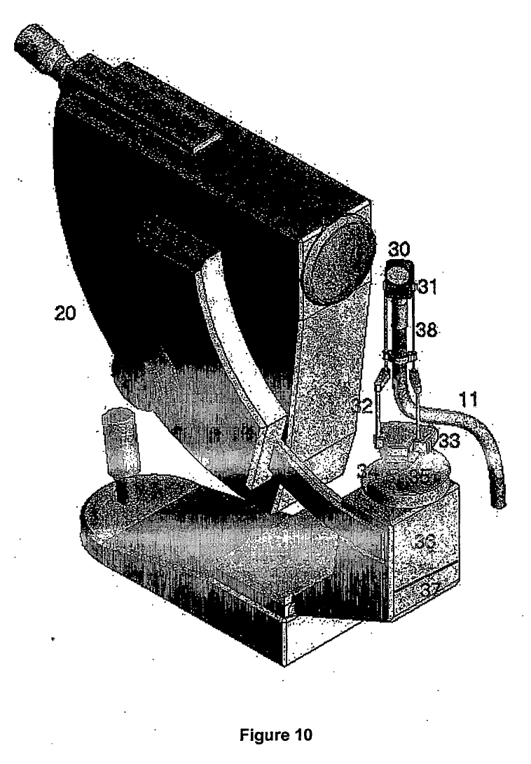

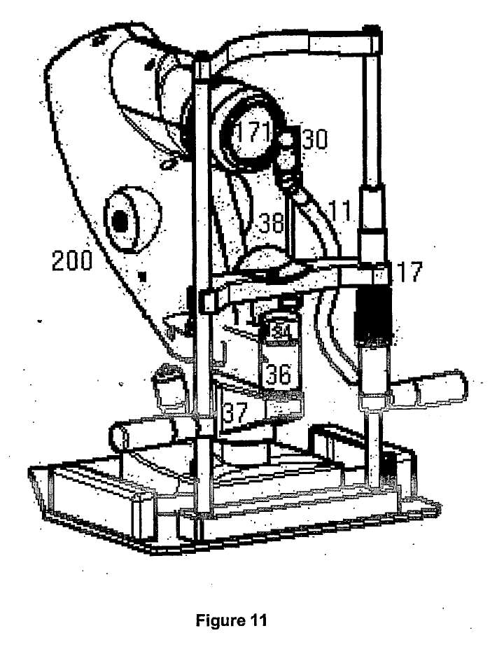

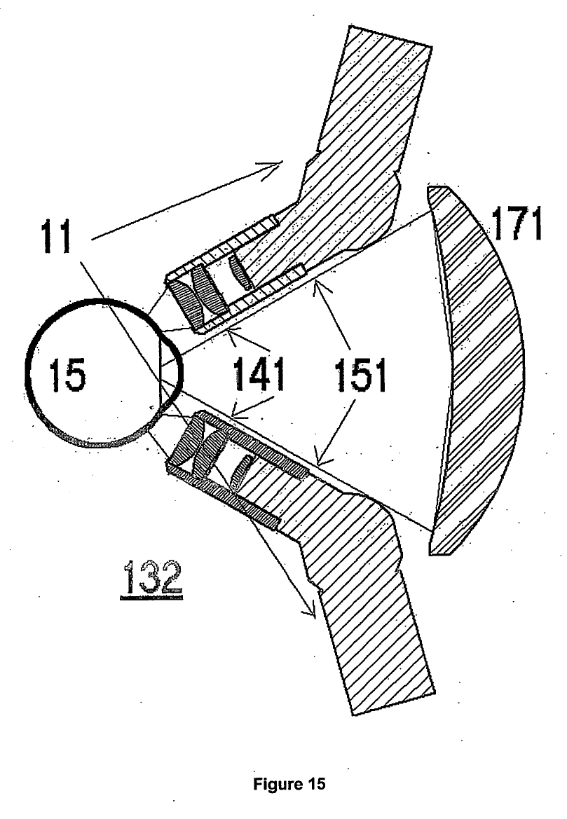

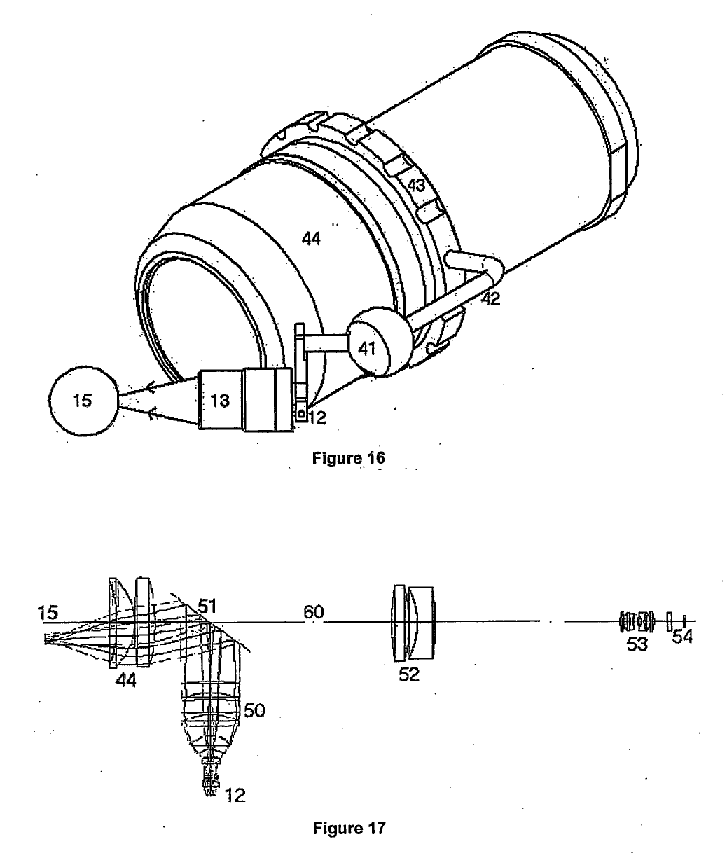

Without losing generality, four of the aforementioned examples are realized by adding to the existing light source of Panoret-1000™ (Medibell Medical Vision Technologies, Ltd.), which is built in accordance with U.S. Pat. No. 6,309,070 (Svetliza, et al.), a focusing element (condenser 13 in FIGS. 2, 5, 6, and 16, condenser 30 in FIGS. 10, 11, and 13; and condenser 141 in FIGS. 8 and 15) that focuses the light energy pattern from the tip of an optical fiber to the eye sclera, and a handling support that holds it (element 16 in FIG. 5, element 38 in FIGS. 10 and 11, elements 41 and 42 in FIG. 16). The focusing elements (condensers) form optics that focus the light from the light source to a light spot on the sclera without touching the sclera. The handling support that holds the focusing elements forms opto-mechanical means for directing the focused beam to a desired position on the eye sclera. These elements are mounted either on a standard fundus camera (FIG. 5 showing a TRC-50X of Topcon, Ltd.), which here provide only the imaging optics for examining the retina, or on a specially designed camera (e.g., FIG. 11). This is unique in the sense that it is the first time that transcleral illumination is applied in a non-contact manner, but it also exemplifies the broad and general use of the disclosed invention.

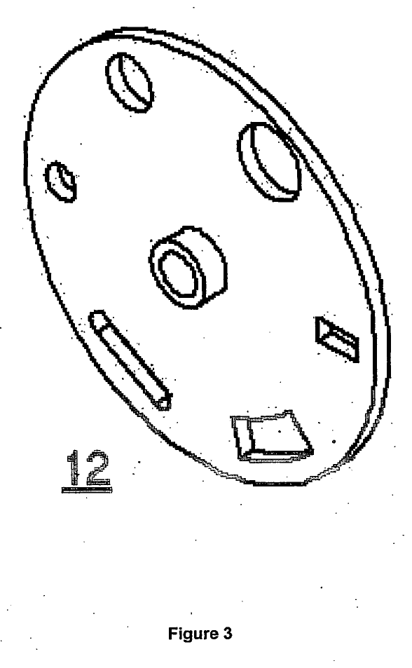

Referring to FIG. 2, condenser 13 includes a thin rotating wheel 12 contiguous to the end of an optical fiber 11. Wheel 12 controls the shape and size of the illumination spot that is projected on the sclera (as illustrated in FIG. 1) in order to adjust it to different eye sizes and eyelid openings. Wheel 12 form means for controlling the shape and means for controlling the size, of the light spot that is created on the sclera by the focused beam. This is done by cutting holes, termed apertures, with the required shape into the thin light-blocking material from which wheel 12 is made (see FIG. 3 for one exemplary embodiment of a wheel). Once such an aperture is centered in front of the end of the optical fiber, fully included in the area that transmits the light, it becomes an object that is imaged on the illumination focal plane, which lies on the sclera, thus shaping the light spot on the sclera.

Condensing lenses 14 that focus the light spot on the sclera can be moved within condenser 13 to provide different alternative focal lengths, i.e., different working distances from the eye. For a given working distance, the efficiency of energy transfer from the optical fiber end to the sclera depends on the diameter of lenses 14 and their distance to the end of the optical fiber. Simple geometrical considerations would show that the further one places condenser 13 from the eye 15, the wider and longer condenser 13 would have to be in order to optimize luminous efficiency. Lenses 14 can optionally be chosen such that each has a different optical power, and different combination of optical powers can serve to control not only the distance of the focused spot from condenser 13 but also its size. Condensing lenses 14 form means for controlling the distance of the optics from the eye.

The part of the illumination system that injects the light into the optical fiber 11, i.e., elements 1 to 10 in FIG. 2 (the light source) can be constructed according to U.S. Pat. No. 6,309,070 (Svetliza, et al.) (the disclosure of which is incorporated herein by reference) and is briefly revisited here. A lamp 1 (by way of example xenon, halogen, or metal-halide lamp, or, any type of filament, arc, or gas lamps) produces a well-defined collimated light beam, with the aid of matching beam-expander optics (a reflector that collects and collimates the light). A hot mirror 2 is placed in the optical path close to the light source to remove ultraviolet (UV) and infrared (IR) components of the light spectral content. The hot mirror 2 and filters wheels 7 and 9, described below, form means for controlling the spectral content of the light from the light source. A condensing lens 3 narrows the beam for practical purposes. A neutral density filter 4 may be inserted to enable a more pronounced light power reduction in the traversing beam. An electro-optical fast shutter 5 (by way of example, a LCP250 scattering liquid crystal polymer shutter by Philips, the Netherlands) controls the amount of light that is transmitted further. The electro-optical fast shutter 5 and the LC shutter control circuit of block 105 described below form means for controlling the intensity of the light in the light spot (i.e., the light energy density). Towards the end of the light path the collimated beam is focused onto an entrance aperture 10 of a fiber optics feeding cable 11, using a short focusing aspheric condensing lens 8.

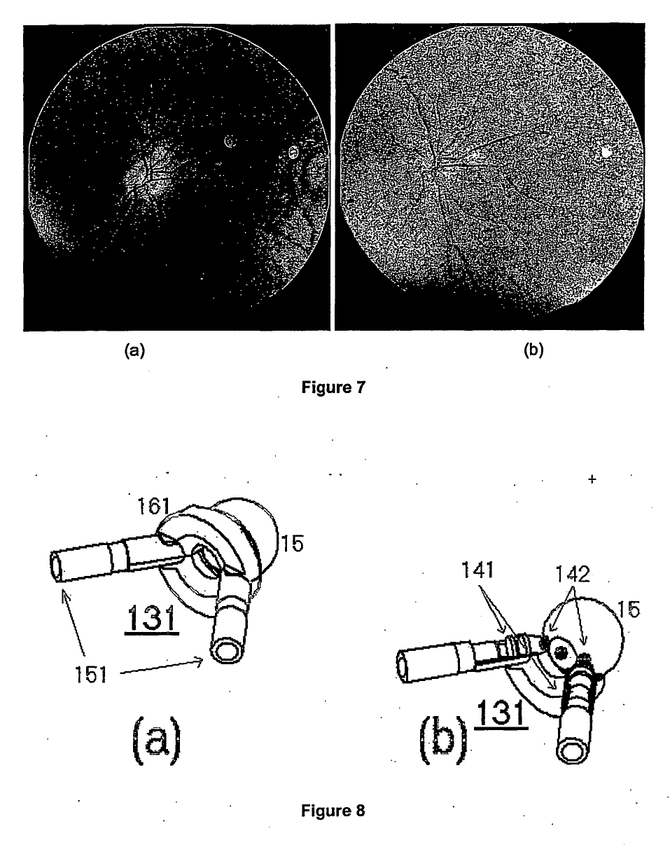

Filters of a rotary wheel 7 may be positioned in the optical path for monochromatic illumination (see a corresponding retinal image in FIG. 7a). Rotary filter wheel 7 has several spaced filters mounted around a disc. Wheel 7 locks in certain positions where one of the interchangeable filters overlaps the entire beam cross section, thus isolating a certain spectral window from the fill “white” content of the beam. This enables a specified spectral band or colored illumination to illuminate the subject. By way of example, the filter wheel may be provided with narrow band pass optical filters 6 and a transparent or empty window. The configuration of the filters wheel is readily understood by one of ordinary skill in the art. Additionally, one embodiment thereof is described in details the referred U.S. Pat. No. 6,309,070 (Svetliza, et al.). When filter wheel 7 is locked in position so that the transparent or empty window extends across the beam cross section, the full power and spectral content of the light beam are allowed for transfer to the next station.

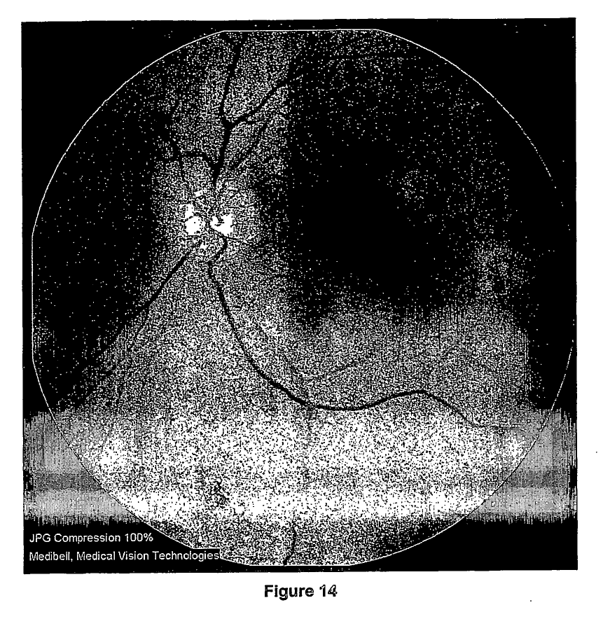

In order to enable color imaging without any loss of the high resolution available from a black and white CCD camera, a second RGBT filter wheel 9 is used in the optical path (see a corresponding retinal image in FIG. 7(b) and FIG. 14). This wheel is divided, by way of example, into 4 partitioned sections, R, G and B sections that equal to one another in size and a fourth section which is a transparent (T) section that is smaller than the R, G and B sections and is used for passing the full original content of the white beam. The dimensions of the transparent section, at a minimum, extend across the cross-section of fiber optic cable entrance aperture 10.

In order to establish the highest achievable duty cycle for each of the three main R, G and B colored sections, RGBT wheel 9 is preferably positioned close to a plane where the beam is narrowed to a minimum (i.e. near the focal plane of fiber optic entrance aperture 10). With wheel 9 thus positioned, the projection of the beam cross-section is small, meaning that the transparent section of the wheel can be at its smallest possible size while still covering aperture 10. This allows the largest duty cycle for the three remaining optically filtered sections, RGB. When RGBT wheel 9 rotates at a speed of one third of the frame rate of the CCD camera, a sequence of definite R, G and B (with a short white) spectral light bursts are transferred to aperture 10 for each revolution of RGBT wheel 9. Each of these R, G and B sequenced light bursts is fully synchronized with one of the consecutive frames of the CCD camera located in the detection channel. This produces R, G and B illuminating images in sequence, each frame of the camera having one color. These images are later composed by the computer into a single colored picture. Thus, every three consecutive monochromatic “colored” images comprise one colored picture. The computer updates these colored pictures at the camera frame rate, each time a new “colored” frame is detected.

Referring again to FIG. 2, when color pictures are no longer required, RGBT wheel 9 is locked in a position where the T section overlaps the beam cross-section, allowing the full impinging light content from lamp 1 to be passed to aperture 10. When locked in this “white” position, the light can be used for specific monochromatic illumination purposes by introducing the appropriate filters into the optical path using filter wheel 7. Further details of elements 1 to 10 in FIG. 2 are described in U.S. Pat. No. 6,309,070 (Svetliza, et al.).

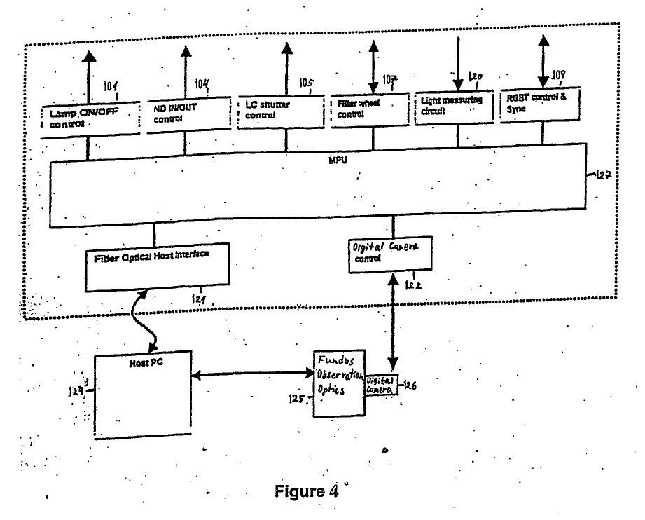

Referring now to FIG. 4, there is shown a block diagram of the computerized controls of the illumination system of FIG. 2 (similar to and described in detail in U.S. Pat. No. 6,309,070, Svetliza, et al.). In the presented system it is realized according to U.S. Pat. No. 6,309,070 (Svetliza, et al.) and is briefly revisited here. The controls include circuitry on a printed circuit board (PCB) designed to control and monitor the optical parts of illumination system in FIG. 2 and interface with a host PC 124.

In block 121, the copper to fiber interface between the PC 124 and the illumination system is provided as a fiber optic interface for signal conversion, with communication of up to 100 Mbit/sec, bi-directional. In block 127, the main processing unit (MPU), which may be, for example an Altera 10 k based type, is in charge of communication with all I/O's and host PC 124. The control algorithms are implemented here, timing and synchronizing all the other controlling elements for controlling the light source, the optics, and the opto-mechanical means.

The filters wheel control is provided in block 107 and drives rotary filter wheel 7 in FIG. 2. An eight channel, 10 bit serial analog to digital converter (ADC) (light measuring circuit) is provided in block 120 for measuring light passing through the light source and for monitoring safe light levels in the light measuring circuit. Block 109 is a RGBT control and sync circuit used to rotate color wheel 9 in FIG. 2 so it is synchronized to the camera frame integration in color mode, and to position the wheel in its transparent sector in monochromatic and angiography test modes. The digital camera 126 in its turn is activated by block 122.

A lamp ON/OFF control circuit in block 101 controls lamp 1 in FIG. 2. This may also be used as an emergency off circuit that reacts to a feedback obtained from a small light detector that sees a small portion of the light beam reaching element 10 in FIG. 2, turning the lamp OFF when the light intensity passes a safety threshold. Neutral density filter 4 (FIG. 2) is inserted or removed by the ND IN/OUT control circuit in block 104 to control light passing therethrough from light source 1. In block 105, there is provided an LC shutter control circuit that controls the fast shutter 5 in FIG. 2 for continuous control of light intensity. The continuous control of light intensity is done as feedback to the intensity of light measured on the camera CCD with the aim of obtaining the strongest signal while avoiding saturation. PC 124 is programmed to pass the feedback from the CCD to MPU 127, which in turn passes the appropriate controlling signals element 105 that controls the LC shutter 5 in FIG. 2. Further details of the computerized control system were already described by U.S. Pat. No. 6,309,070 (Svetliza, et al.).

In an alternative embodiment of the patent, the aforementioned lamp (element 1 in FIG. 2) is replaced by an array of many smaller light sources (not shown). By way of example, laser diodes or light emitting diodes (LED) are arranged on a spherical surface with their principal light emission axis perpendicular to that surface. The precise arrangement of the light sources is within the skill of the ordinary artisan. As a result, most of the light energy that is emitted by these diodes is concentrated at the center of the sphere, creating a small light spot that corresponds in size to the light-emitting gap in a single diode chip but has the energy that is the sum of the energies emitted from all the diodes together. Collimating optics is applied to each one of the diode sources, in a manner within the skill of the ordinary artisan, bringing the size of the light spot at the center of the sphere down to an order of magnitude of hundreds of microns.

The spectral characteristics of the diodes array are determined by the choice of diodes put in the array and their emission intensity is electronically controlled by adjusting the electric potential on the diode chip. Hence, the optics corresponding to a diode array-based system is described by FIG. 2 without elements 4 to 9 in and its controls by FIG. 4 without elements 104 to 109. Moreover, the small dimensions of the diode chips make it possible to attach the diode array illumination source directly to condenser 13 in FIGS. 2, 5, 6, and 16, to elements 131 in FIGS. 8 and 9, to element 30 in FIGS. 10, 11, and 13, or to elements 132 in FIG. 15, requiring an appropriate adjustment of element 12 and lenses 14 and 141, respectively. Alternatively, the entrance aperture 10 in FIG. 2 can be centered at the focus point of the diode array, efficiently transmitting the light into the optical fiber 11. The numerical aperture of the optical fiber determines the maximal angular opening of the spherical segment on which the diodes are arranged. Accordingly, the larger the radius of the sphere, the greater the number of diodes arranged on it can be.

### EXAMPLE 1

### Illumination Focusing Element Attached to a Chin Rest

FIG. 5 shows an example of the present invention in conjunction with the imaging optics of a standard fundus camera (by way of example Topcon's TRC-50X) that operates at a distance of approximately 5 cm from the eye cornea. In the camera of FIG. 5, the elements that focus the light on the eye sclera are coupled to a chin rest system that fixes the patient's face and eye position relative to the projected light and with the possibility of directing the orientation of the eye. Optical fiber 11 (see also FIG. 2) conveys the light from the light source to condenser 13, which is supported by the adjustable arm 16 that gives a full freedom to focus the light spot to the right position on the patient's eye sclera as in FIG. 1. Focusing of the spot takes place while the patient's head is resting on the chin rest 17. When illuminating the sclera with condenser 13 (see FIG. 2), the optics of the TRC-50X (by way of example) conveys the image of the retina through optical adapter 18 to CCD camera 19, which is connected and activated by the controls shown in FIG. 4.

Arm 16 is devised in a way that it allows moving condenser 13 from optimally illuminating one eye to optimally illuminate the other eye. Arm 16 forms means for efficiently switching the focused beam from eye to eye. Alternatively, a system could be devised within the skill of the ordinary artisan to have two sets each consisting of elements 16 and 13, symmetrically positioned to fit for the two eyes simultaneously. In FIG. 6, two optical fibers 11 convey the light to two condensers 13 separately and two elements similar to element 10 in FIG. 2 are mounted on platform 90 with a mechanism 100 that can center a selected fiber in front of the central illumination axis 110, shown by a broken line. Moving platform 90 switches between injecting the illuminating light into one or the other of the fibers. This is done either manually or by an electric motor 100 that is be controlled manually or electronically.

FIGS. 7(a) and 7(b) show examples of retinal images acquired with the system in FIG. 5 when connected to the controls shown in FIG. 4. FIG. 7(a) is a monochromatic “red-free” image of a patient's right eye retina, while FIG. 7(b) is an RGB color image of the same retina. The images were acquired without dilating the patient's pupil, which had a diameter of approximately 2 millimeters. The nasal portion of the retina that is seen here through a 2 millimeters pupil is quite remarkable and illustrates the advantages of transcleral illumination as discussed earlier herein.

### EXAMPLE 2

### Illumination and Focusing Elements Encased in a Device that Positions the Patient's Eye Appropriately for Transcleral Illumination

Further, in yet another embodiment of the invention, optical fiber 11 can be split into two, leading to optics 131 that illuminate the sclera simultaneously both on the nasal and on the temporal sides of the eye. FIGS. 8(a) and 8(b), illustrate a device that encases optics 141 to focus the light illumination spots 142 that originate from optical fiber ends 151 on the sclera of eye 15. Device 131 is coupled to a chin rest, and the two optical fiber ends stem from a single optical fiber (e.g., optical fiber 11 in FIG. 2) that is split into two (see e.g., FIG. 9) by a well-known technology. Head positioning on the chin rest is done in a way that the patient approaches it with the eye first, to touch ring 161 externally to the eyelids, and only afterwards adjusts the chin rest to support the head for the acquisition. The observation and imaging system then moves independently until a good view of the retina is obtained. Afterwards, the patient moves with the other eye to fit onto ring 161, or, alternatively, optics 131 is moved to illuminate the other eye.

FIG. 9 illustrates an alternative embodiment, in which two optics 131 are attached to the chin rest 17 to fit the two eyes of the patient simultaneously. This way the patient does not need to move his or her face while the observation and imaging system is switching from looking in one eye to the other one. In this case, two optical fibers 11 are split into two, leading to two optics 131. The two optics 131 are mounted on mechanism 133 that serves for adjusting the distance between them to fit the face structure of the patient. Switching between illuminating the left and the right eye is done (by way of example) by mechanism 100 in FIG. 6 as described in Example 1 above.

In an alternative embodiment of this example, a device similar to 131 could serve to illuminate the sclera only from the temporal side, waiving the need to take the nose of the patient into account. It requires however either a mechanism to rotate it 180 degrees when switching from eye to eye, or, two optics, one for each eye and a set-up similar to the one in FIG. 6 that includes two optical fibers and a switching mechanism to switch between the illumination of one eye and the other one.

The methods and systems described in this example reassure the appropriate positioning of the illumination spots on the eye sclera, independent of the imaging optics, and form opto-mechanical means for directing the focused beams to desired positions on the eye sclera.

### EXAMPLE 3

### Illumination Focusing Element Attached to the Same Moving Platform as the Optical Imaging System Apart from Rotation

FIGS. 10 and 11 show a third example of the present invention. In FIG. 10, the present invention is implemented in conjunction with the imaging optics of a standard fundus camera that operates at a distance of approximately 5 cm from the eye cornea. In the system of FIG. 10, the elements that focus the light onto the eye sclera are coupled to the optical system that is used to observe the interior of the eye in a way that whenever the optics is properly positioned to observe the interior of the eye, the illumination light spot is properly focused at the right position on the eye sclera. In FIG. 11, the present invention is implemented with an imaging optics that was especially designed to exploit the advantages of non-contact transcleral illumination. As in the system of FIG. 10, in FIG. 11, the elements that focus the light onto the eye sclera are coupled to the optical system that is used to observe the interior of the eye in a way that whenever the optics is properly positioned to observe the interior of the eye, the illumination light spot is properly focused at the right position on the eye sclera. In both figures, optical fiber 11 (see also FIG. 2) conveys the light from the light source (by the way of example, elements 1 to 10 in FIG. 2) to the focusing element 30 that is supported by a rotating arm 38 that is coupled by an axis base 35 to the same platform 37 that carries the optical imaging system 20. A set of joints (elements 31 to 34) provides all the necessary degrees of freedom to ensure that the imaging system and the focused light spot will be properly positioned simultaneously. The swivel element 31 allows tilting of element 30 in order to optimize the optical path to the sclera, avoiding the upper eyelid. Element 32 adjusts the height of element 30 and element 33 adjusts the distance relative to the optical imaging system. In order to allow imaging from different angles relative to the eye, the rotation axis 34 is coupled to the carrying platform basis 37 but not arm 36 that carries optical imaging system 20 or 200.

The imaging system 200 in FIG. 11 was devised specially to function together with non-contact transcleral illumination. Different from the imaging system 20 in FIG. 10 and all other standard fundus cameras, system 200 does not include a light source and optics that direct the illumination into the eye but consists only of imaging optics.

The appearance of the system in FIG. 11 corresponds to a typical arrangement upon acquiring a retinal image of the right eye. During examination and photography the patient rests the head on chin rest 17. The operator then directs the imaging system until the pupil of the eye coincides with the pupil of the imaging optics and the retina fills the field of view of the camera. The present invention reassures that concomitantly the illumination light spot reaches its optimal position on the eye sclera and enough light fills the interior of the eye, reflecting a good retinal image on the camera detector, allowing focusing, final adjustments, and image recording. In order to acquire an image of the other eye, the light focusing element 30 is rotated around axis 34 and is symmetrically positioned on the other side of the patient's face.

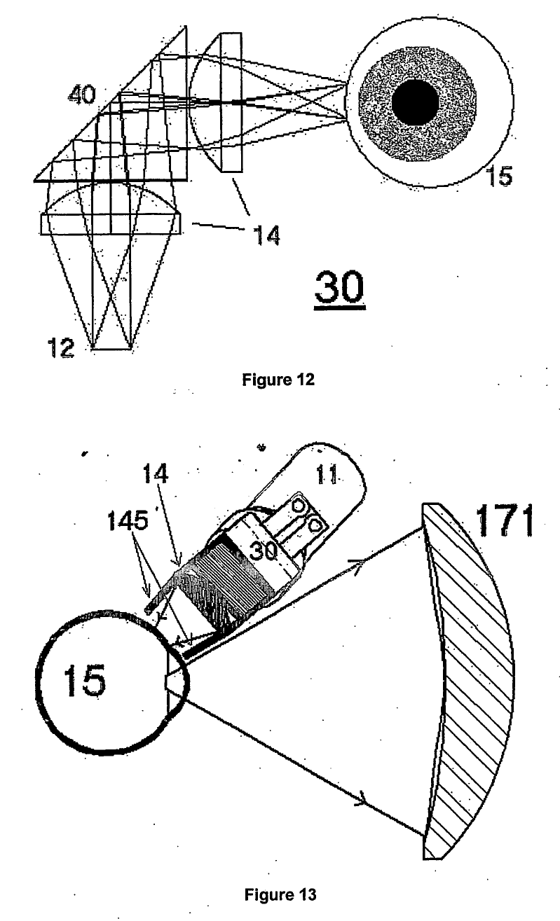

The design of the focusing element 30 yields optical properties that are similar to the optical properties of element 13 in FIGS. 2, 5, 6 and 16 in an arrangement (see FIG. 12) that reduces its horizontal length and permits free rotation from side to side without colliding with the forefront elements of the optical imaging system (see FIGS. 10 and 11).

FIG. 12 shows an embodiment of focusing element 30 that serves the purpose of minimizing the horizontal length of the element to support switching the illumination from eye to eye without colliding with the imaging optics (see FIGS. 10 and 11). This embodiment of the present invention enables an efficient switch from eye to eye. Light enters focusing element 30 through wheel 12 (as described in conjunction with FIG. 2) to which the optical fiber bundle 11 of FIG. 2 (not shown) is connected. Lenses 14 focus the light on the sclera of eye 15, while prism 40 serves for folding the light beam.

As not all optical systems that serve for observing and imaging the interior part of the eye are optimized to deal with the angular content of light that may reach their front lenses upon transcleral illumination, an extra shield can be attached to condenser 30 in order to block the optical observation system from seeing that light. Without loosing generality, FIG. 13 illustrates an exemplary embodiment, in which a thin light-blocking foil 145 extends from condenser 30 as much as possible towards the eye without touching it, along a path that would block light that is scattered from the sclera of eye 15 without entering the field of view of the observation optics 171. Alternatively, and within the skill of the ordinary artisan, the extra shield could be a cone made of a thin light-blocking material that would fit to include the light beam that is focused by condenser 30 and it shall extend to reach the eyelids, without touching the eye. The extra shield, described here in two embodiments, can be formed in alternative ways within the skill of the ordinary artisan. The extra shield forms a final element of the optics with light blockers that extend to the eyelids and prevent light that is reflected or scattered from the surface of the eye from reaching the observation and imaging optics.

FIG. 14 shows an example of a retina image acquired with the system in FIG. 11 when connected to the controls shown in FIG. 4.

An alternative realization of the concept described in this example could include a duplication of an element similar to element 13 in FIGS. 2 and 5 (see also FIG. 8) so that both the nasal and the temporal sides of the sclera would be illuminated simultaneously in order to optimize the illumination of the eye for different angles of observations. In such a case, optical fiber 11 is split into two (see FIG. 9), and the sizes of all the elements are designed to avoid collisions with the observation optics and with the nose of the patient.

FIG. 15 illustrates optics that consists of lenses 141 embedded in a casing that connects optical fibers 11 via a 45 degrees bent connector. The sizes of all elements are such that the optics neither collides with the patient's nose nor enters the field of view 151 or imaging system 171.

### EXAMPLE 4

### Illumination Focusing Element Attached to the Optical Imaging System

FIG. 16 shows a fourth example of the present invention in which the elements that focus the light onto the eye sclera are coupled to the optical system that is used to observe the interior of the eye in a way that whenever the optics are properly positioned to observe the interior of the eye, the illumination light spot is properly focused at the right position on the eye sclera.

The focusing element 13 is here held by an arm 42 that is connected to a ring 43 that is fitted to a tube that holds the front optics 44 of the optical imaging system. In order for the system to serve for both eyes, ring 43 can rotate around the imaging-optics to be symmetrically positioned on either side of the central optical axis of the imaging optics. A mechanical joint 41 serves as a swivel to allow aiming the focused light spot to the appropriate position on the sclera of eye 15, right above the pars plana. Illumination light is fed into this system via fiber optic bundle 11 (see FIG. 2) that connects to wheel 12 with all its properties as mentioned in relation to example 1.

In an additional embodiment of the presented example elements 12, 13, 41, and 42 can be duplicated to be attached symmetrically on both sides of optics 44 thus waiving the need to use rotating element 43 in order to adapt the system to the two eyes. Two optical fibers as illustrated in FIG. 6 are then required with a mechanism to switch between them when switching between the two eyes.

In comparison to example 3, this system has the advantage of being adaptable to any fundus optical imaging system, independent of the platform that carries it. One drawback is that when rotating the optical system in order to observe different portions of the interior of the eye, the illumination light spot moves along with it away from the optimal position on the sclera.

### EXAMPLE 5

### Illumination Sharing Optics with the Imaging System

FIG. 17 shows the optical set up for focusing a light spot on the sclera of eye 15 along with the optical elements composing another example of a retinal imaging system according to the present invention in which part of the imaging optics is shared with the illumination optics to create the required illumination patterns at predetermined distances from the center of the optical axis so that they fall on the eye sclera at the required distances from the limbus. The dark line marks the central optical axis 60 that goes through the pupil of eye 15 upon imaging the retina. Lens assembly 44 creates an intermediate image of the retina. Lens assembly 52 serves for focusing and assembly 53 resizes the image to fit on the camera detector 54.

In order to focus the illuminating light onto the right location on the sclera of eye 15, at about 12 millimeters from the center of the pupil, a very thin (pellicle) beam splitter 51 is used to direct the light off axis from the light source through the front lens assembly 44 without distorting the image. The light is introduced by an optical fiber bundle through wheel 12, which has similar properties to those described in example 1 in reference to FIG. 2. The beam properties are then adjusted by a set of lenses 50 in such a way that when passing through assembly 44, the beam is focused on the right position.

In order to switch the illumination spot from one side of the pupil to the other one, the beam splitter 51 is rotated. In this example, the required rotation is about 10 degrees. Moving the illumination spot from one side to the other is necessary when switching the photographed eyes or when rotating the optical imaging system for observing different regions inside the eye.

By electronically controlling the position of element 51, it is possible to optimize automatically the position of the illuminating spot relative to the central axis A in each position of the camera. This is done by putting detectors on the rotation axis (by way of example, the rotation axis of arm 36 in FIG. 10) of the imaging system in order to detect the rotation angle of the camera, as well as putting detectors on the carrying platform (by way of example, element 37 in FIG. 8) in order to detect which eye the camera is observing. The beam splitter 51 and such detectors form means for controlling the angle relative to the central optical axis of the eye at which the center of the focused beam reaches the sclera, thus controlling the distance of the light spot on the sclera from the limbus on one side and from the corner of the eye on the other side, and accordingly adjusting to an optimal position of the light spot relative to eye size.

In order to avoid optical noise that may result from specular reflections of illuminating light coming from assembly 44, one light polarizer can be inserted between elements 12 and 51 and another one producing polarization perpendicular that of the first polarizer between elements 51 and 52.

In an alternative set up, beam splitter 51 can be replaced by a toroid-shaped mirror and an optical design in which the light is shined in a toroidal shape on the mirror before being focused into a spot by assembly 44. The design and placement of these elements are considered to be within the skill of the ordinary artisan. The path of the imaging beams then goes through the hole in the mirror on its way from the interior of the eye to the image detector. This set up is useful for overcoming the loss of illumination energy and imaging signal that occur when using a beam splitter since beam splitters transmit part of the light and reflect the other part.

Example 5 has the advantage over the previous examples in being compact and allowing electronic optimization of the illumination light spot position on the eye sclera. It suffers from the fact the illumination power is not efficiently used because of the losses involved upon folding it inside the imaging optics system. It also has the drawback that it cannot be added to an existing imaging system but requires a combined design of the imaging system together with the illumination set up.

Having described the invention with regard to certain specific embodiments thereof, it is to be understood that the description is not meant as a limitation, since further modifications may now suggest themselves to those skilled in the art, and it is intended to cover such modifications as fall within the scope of the appended claims.

What is claimed is:

1. A method for illuminating the interior of an eye through the sclera of the eye, comprising
focusing a light beam on the sclera by focusing optics while maintaining the focusing optics out of contact with the sclera.

2. The method of claim 1. wherein said step of focusing is carried out with opto-mechanical means operative to direct the focused light beam to a desired position on the sclera.

3. A system for ophthalmic illumination of the interior of the eye of a patient through the sclera of the eye without touching the eye comprising:
a light source;
illumination optics that focus the light from the light source to a light spot on the sclera without touching the sclera; and
opto-mechanical means for directing the focused beam to a desired position on the eye sclera.

4. The system of claim 3, wherein said light source is a lamp.

5. The system of claim 3, wherein said light source is composed of a plurality of small light sources.

6. The system of claim 3, further comprising means for controlling the shape of the light spot that is created on the sclera by the focused beam.

7. The system of claim 6, in which the shape of the light spot is one of: circular; elongated; and slit-like.

8. The system of claim 7, in which the light spot is elongated and is oriented with a longer axis parallel to the eyelids such that the amount of light falling on the sclera without hitting the eyelids is maximized, and at least part of the light falls exactly at an optimal position on the sclera.

9. The system of claim 3, further comprising means for controlling the size of the light spot that is created by the focused beam on the sclera.

10. The system of claim 3, further comprising means for controlling the distance of the optics from the eye.

11. The system of claim 3, further comprising means for controlling the angle relative to the central optical axis of the eye at which the center of the focused beam reaches the sclera, thus controlling the distance of the light spot on the sclera from the limbus on one side and from the corner of the eye on the other side, and accordingly adjusting to an optimal position of the light spot relative to eye size.

12. The system of claim 3, further comprising means for controlling the angle at which the center of the focused beam reaches the sclera.

13. The system of claim 3, further comprising observation and imaging optics for observing and imaging portions of the eye illuminated by the illumination optics and opto-mechanical means.

14. The system of claim 13, wherein the illumination optics comprise a final element with light blockers that extend to the eyelids and prevent light that is reflected or scattered from a surface of the eye from reaching the observation and imaging optics.

15. The system of claim 3, further comprising means for controlling spectral content of the light from the light source.

16. The system of claim 3, further comprising means for controlling the intensity of the light in the light spot.

17. The system of claim 3, further comprising means for timing all controls.

18. The system of claim 3, further comprising programmed controls that are automatically adjustable according to feedback obtained from a light detector.

19. The system of claim 3, further comprising means for efficiently switching the focused beam from eye to eye.

20. The system of claim 3, further comprising optics for focusing two light beams on the eye sclera simultaneously, one on the nasal side of the eye and the other one on the temporal side of the eye.

21. The system of claim 3, further comprising optics for focusing two light beams on the sclera of both eyes.

22. The system of claim 3, further comprising optics for focusing four light beams on the eye sclera of both eyes, two beams for each eye, one on the nasal side and the other one on the temporal side.

23. The system of claim 3, wherein the light source and illuminating optics are coupled to a chin rest system that fixes a patient's face and eye position relative to the light spot and with the possibility of directing the orientation of the eye.

24. The system of claim 3, wherein the light source and the optics are coupled to an optical observation system that is used to observe and image the interior of the eye in a way that whenever the optics is properly positioned so to observe the interior of the eye, the light spot is properly focused at a desired location on the eye sclera.

25. The system of claim 24, wherein said optical observation system couples all degrees of freedom between said optical system and the light source, apart from rotation, so that said optical observation system can observe the interior of the eye from different angles while the focused light spot remains positioned appropriately on the eye sclera.

26. The system of claim 3, further comprising an optical fiber that is coupled to convey light from said light source to optics that lie close to the patient's eye and focus the light on the sclera of the eye.

27. The system of claim 26, wherein said optics are coupled to a chin rest system that fixes the patient's face and eye position relative to the light spot and with the possibility of directing the orientation of the eye.

28. The system of claim 26, wherein said optics are coupled to an optical observation and imaging system that is used to observe and image the interior of the eye in a way that whenever the optics is properly positioned so to observe the interior of the eye, the light spot is properly focused at the desired position on the eye sclera.

29. The system of claim 28, wherein said system couples all degrees of freedom between the optical observation and imaging system and the light source, apart from rotation, so that the optical observation and imaging system can observe the interior of the eye from different angles while the light spot remains positioned appropriately on the eye sclera.

30. The system of claim 3, further comprising two optical fibers that are coupled to convey light from said light source to two optics, which focus the light on the eye sclera at the nasal and temporal sides simultaneously.

31. The system of claim 30, wherein said optics are coupled to a chin rest system that fixes the patient's face and eye position relative to the light spots and with the possibility of directing the orientation of the eye.

32. The system of claim 30, wherein said optics are coupled to an optical observation and imaging system that is used to observe and image the interior of the eye in a way that whenever the optics is properly positioned so to observe the interior of the eye, the light spots are properly focused at the desired positions on the eye sclera.

33. The system of claim 32, wherein said system couples all degrees of freedom between the optical observation and imaging system and the light source, apart from rotation, so that the optical observation and imaging system can observe the interior of the eye from different angles while the light spots remain positioned appropriately on the eye sclera.

34. The system of claim 3, further comprising two optical fibers that are coupled to convey light from said light source to two optics, one for each one of the patient's eyes, which focus the light on the sclera of the eyes.

35. The system of claim 3, further comprising four optical fibers that are coupled to convey light from said light source to four optics, two for each one of the patient's eyes, which focus the light on the eye sclera at the nasal and temporal sides simultaneously.

36. The system of claim 3, further comprising an observation and imaging optics that shares components with said illumination optics and creates the focused light spot at a predetermined distance from the center of an optical axis so that the focused light spot impinges on the eye sclera at an optimal location for light penetration.

37. The system of claim 36, wherein said system creates at least two spots of focused light on the sclera of the eye at spaced points on a circle around a central optical axis at optimal locations for light penetration, and said system further comprises a control mechanism that selects the best illumination spot for each position of the optical observation and imaging system.
